(19) United States
(12) Patent Application Publication
(10) Pub. No.: US 2007/0288022 A1
(43) Pub. Date: Dec. 13, 2007

(54) Bone reconstruction plate with improved fatigue resistance

(75) Inventor: Christian Lutz, Monkeberg (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Trauma GmbH, Schonkirchen (DE)

(21) Appl. No.: 11/784,773

(22) Filed: Apr. 9, 2007

Related U.S. Application Data
(60) Provisional application No. 60/794,608, filed on Apr. 25, 2006.

(51) Int. Cl. A61B 17/80

(52) U.S. Cl. 606/69

(57) ABSTRACT

A bone reconstruction plate is formed of a longitudinally extending plate defining one or more attachment bores. Within one or more of the attachment bores, one or more ridges are disposed around at least a portion of the interior of the one or more attachment bores, whereby the one or more ridges operate to provide increased resistance to fatigue.

This application claims the benefit of the filing date of U.S. Provisional Patent Application No. 60/794,608 filed Apr. 25, 2006, the disclosure of which is incorporated herein by reference.

### BACKGROUND OF THE INVENTION

The present invention relates to bone reconstruction plates, and more particularly to bone reconstruction plates operable to provide improved resistance to fatigue failure.

### SUMMARY OF THE INVENTION

In the field of orthopedic devices, bone reconstruction plates are used to immobilize a fracture or to correctly position a bone or bone fragments in a reconstructive procedure. The bone reconstruction plate further operates to carry loading which is ordinarily placed upon the bone during the period of the bone fragment's healing.

The bending stiffness of the reconstruction plate should lie within a predefined range of the particular bone to which it is attached. If the bending stiffness of the reconstruction plate is too great, the bone under repair is insufficiently loaded, and osteoporosis of the bone may occur. If the bending stiffness of the reconstruction plate is too small, bone resorption/dissolution can occur from movement and excessive force being applied to the bone fragments.

Within the appropriate range of bending stiffness, the bone reconstruction plate should further possess a minimum resistance to fatigue fracture in order to ensure against mechanical failure of the plate. Once implanted, the patient's movement will produce tensile, compressive, and shear stresses upon the plate, and the plate must be capable of withstanding such stresses. Ensuring a high resistance to fatigue failure becomes more critical for bent bone plates, as the bending process typically weakens (i.e., lowers) the plate's fatigue resistance.

### SUMMARY OF THE INVENTION

It may be desirable to provide a bone reconstruction plate which is operable to provide improved fatigue resistance.

This need may be met by a bone reconstruction plate according to the independent claims.

In one embodiment of the invention, a bone reconstruction plate is formed of a longitudinally extending plate defining one or more attachment bores. Within one or more of the attachment bores, one or more ridges are disposed around at least a portion of the interior of the one or more attachment bores, whereby the one or more ridges operate to provide increased resistance to fatigue failure around the holes of the bone reconstruction plate.

In an optional embodiment, the one or more ridges form a thread pattern for engaging a screw or other attaching means within the attachment bore, the screw or other attachment means operable to secure the bone reconstruction plate to the bone.

In a first exemplary embodiment of the invention, the bone reconstruction plate measures 76 mm×8.8 mm×2 mm, the bone reconstruction plate including six 3.4 mm diameter holes and constructed from stainless steel 1.4441. In this embodiment, the bone reconstruction plate is operable with a range of tensile stress between 774 MPa and 807 MPa, a range of compressive stress between 532 MPa and 539 MPa, and a range of shear stress between 12 MPa and 45 MPa.

In a second exemplary embodiment of the invention, the bone reconstruction plate measures 72 mm×10.2 mm×3 mm, the bone reconstruction plate including six 4.4 mm diameter holes and constructed from stainless steel 1.4441. In this embodiment, the bone reconstruction plate is operable with a range of tensile stress between 354 MPa and 366 MPa, a range of compressive stress between, 303 MPa and 320 MPa, and a range of shear stress between 7.4 MPa and 23.6 MPa.

In a third exemplary embodiment of the invention, the bone reconstruction plate measures 94 mm×12 mm×4.1 mm the bone reconstruction plate including six 5.6 mm diameter holes, and is constructed from stainless steel 1.4441. In this embodiment, the bone reconstruction plate is operable with a range of tensile stress between 121 MPa and 124 MPa, a range of compressive stress between, 112 MPa and 114 MPa, and a range of shear stress between 2.6 MPa and 12.4 MPa.

These and other aspects of the present invention will become apparent from and elucidated with reference to the embodiment described hereinafter.

### BRIEF DESCRIPTION OF THE DRAWINGS

An exemplary embodiment of the present invention will be described in the following, with reference to the following drawings:

FIG. 1 illustrates an exemplary embodiment of a bone reconstruction plate operable to provide improved fatigue resistance in accordance with the present invention;

FIGS. 2A-2C illustrate Tables I, II and III showing the magnitude of tensile, compressive and shear stresses for the unimproved and improved bone reconstruction plates.

For clarity, previously-identified features retain their reference numerals in subsequent drawings.

### DETAILED DESCRIPTION

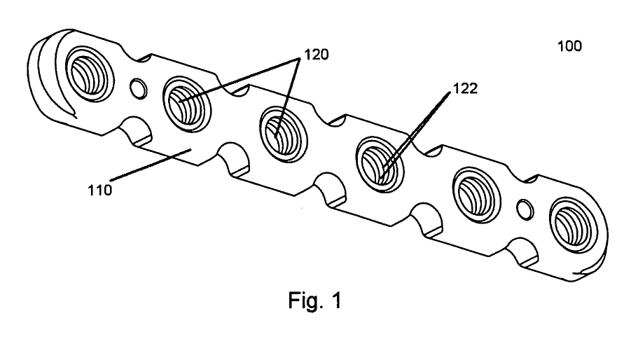

FIG. 1 illustrates an exemplary embodiment of a bone reconstruction plate 100 operable to provide improved fatigue resistance in accordance with the present invention. The bone reconstruction plate 100 includes a longitudinally extending plate 110, within which one or more attachment bores 120 are defined. One or more ridges 122 are disposed within one or more of the attachment bores 120, the ridges 122 operate to provide improved fatigue resistance to the bone reconstruction plate 110 in the area of the attachment bores 120 where fatigue failure is most likely to occur. The one or more ridges 122 may be formed in segments which extend only partially around the inner circumference of the attachment bores 120, or the ridges 122 may be of an annular shape which extends around the entire inner circumference of the attachment bore 120. In a separate alternative embodiment, the ridges 122 may be arranged in a threaded pattern (e.g., a helix) operable to engage an attachment screw used to secure the bone reconstruction plate to the bone. In a particular embodiment, the ridges 122 are formed from the same material as the bone reconstruction plate (e.g., 1.4441 (annealed), pure Titanium (TiCP)). The dimensions of the ridges 122 are selected such that the desired fatigue resistance is met, and can be arrived at empirically by testing the resulting plate for its resistance to tensile, compressive and shear stresses. In a particular embodiment further illustrated below, the one or more ridges 122 defines a thread pattern operable to engage an attachment screw, the attachment screw used to secure the bone reconstruction plate to a bone. In such an instance, the ridges operate to provide the aforementioned increased resistance to fatigue failure, as well as providing a means of securing the attachment screw to the bone reconstruction plate within the attachment bore.

In a particular embodiment of the invention, the bone reconstruction plate 100 is manufactured from a material composition of surgical stainless steel 1.4441 having 0.2% yield strength of 690 N/mm² minimum, an ultimate tensile strength of 860-1100 N/mm² tensile strength and a modulus of elasticity in tension of 186′400 N/mm². In another embodiment, Titanium (TiCP) having 0.2% yield string of 190 N/mm², an ultimate tensile strength of 490-690 N/mm² tensile strength, and a modulus of elasticity in tension of 114′000 N/mm² is employed.

### EXAMPLES

The above-described bone reconstruction plates were manufactured according to the features described above, and measured for tensile, compressive and shear stresses in the following examples. The stress parameters were also compared against bone reconstruction plates which did not employ one or more ridges 122 within the attachment bores 120

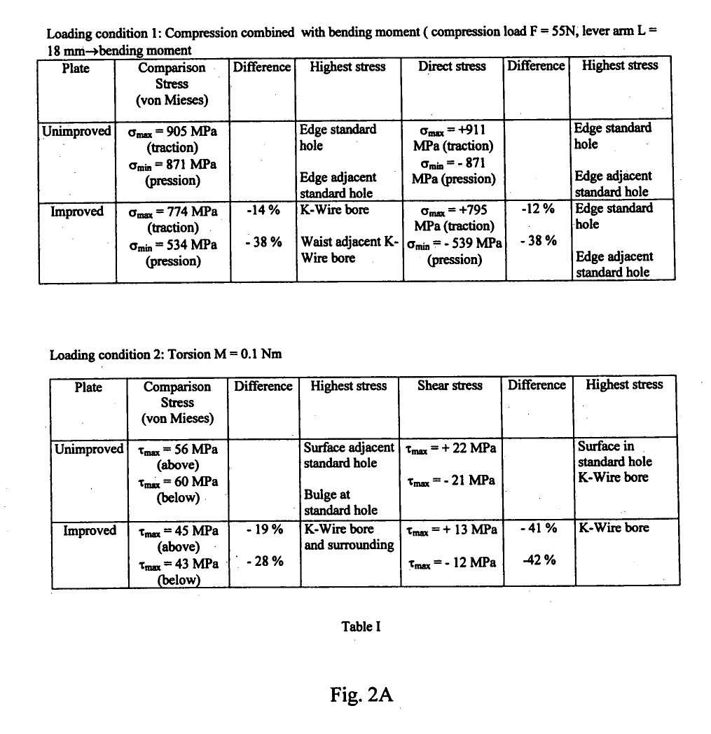

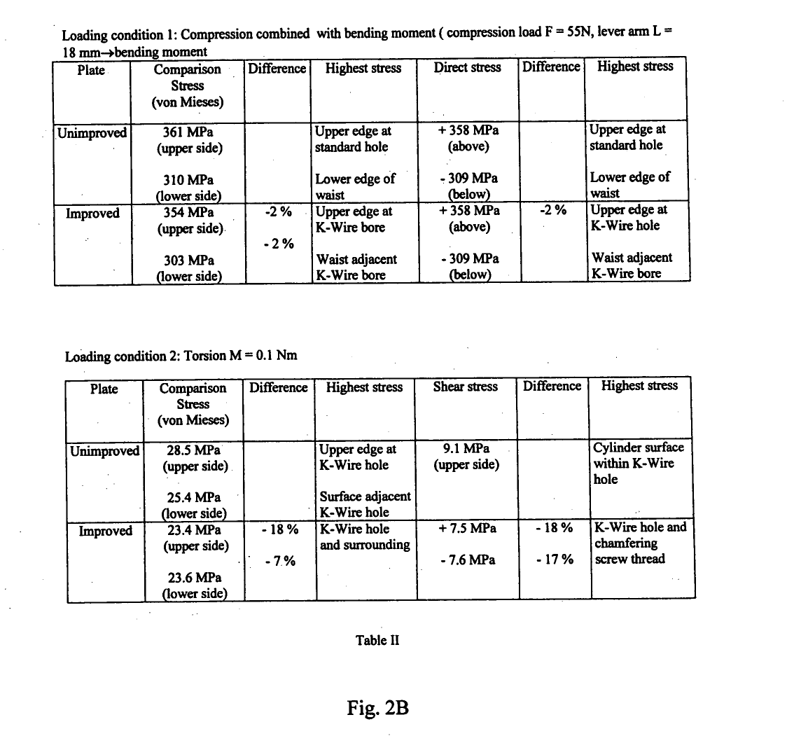

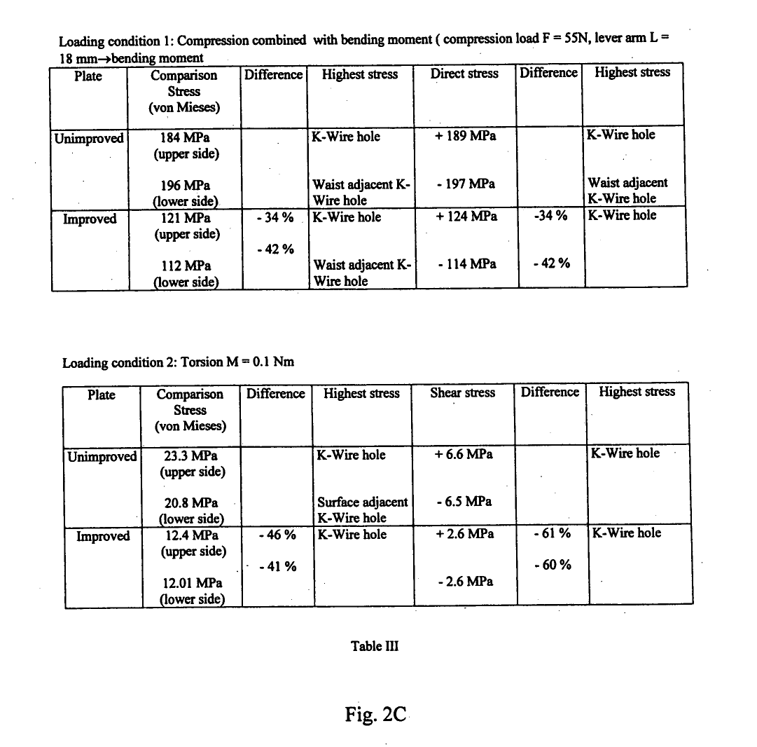

In the comparison, two load cases were tested. In the first load test, a combined compression and bending load test was performed, whereby a compression load of 55 N at a bending moment of 1 N was applied, the resulting force being equivalent to a 55 N force applied on a lever arm of 18 mm. The load test was based upon a simplified fracture model with a cylinder diameter of 35 mm simulating the proximal tibia shaft part. The lever arm corresponds to the distance from the cylinder axis to the middle of the bone reconstruction plate. Equivalent and principle stresses were analyzed. In the second load test, a torsion load was applied, in which a torsion moment of 0.1 Nm was used. Equivalent and principle stresses were also analyzed. In each of the tests, the plates were constructed from stainless steel 1.4441. Tensile, compressive, and shear stresses of the improved bone reconstruction plate were measured relative to bone reconstruction plates without attachment bore ridges 122, negative percentages representing lower measured stress, and accordingly, higher stress resistance for the improved bone reconstruction plates. Stress measurements are provided in terms of a Von Mises stress in accordance with procedures described by BML 06-014, 06-016 06-019. FIGS. 2A-2C illustrates Tables I, II and III showing the magnitude of tensile, compressive and shear stresses for the unimproved and improved bone reconstruction plates.

### One-Third Tubular Bone Reconstruction Plate

Two versions of a one-third tubular bone reconstruction plate measuring 76 mm×8.8 mm×2 mm and having six 3.4 mm outer diameter attachment bores (compatible with 3.0 mm diameter screws) were each tested using the two load cases as described above. As shown in Table I of FIG. 2A, the improved plate, which includes ridges 122 arranged in a threaded pattern for engaging a 3.0 mm diameter screw within each of the attachment bores, exhibited between 774 MPa and 795 MPa of tensile stress and between 534 MPa and 539 MPa of compressive stress, these ranges representing generally 14% and 38% lower stress factors than the unimproved bone reconstruction plate. Regarding the second load test, the improved plate exhibited between 12 MPa and 45 MPa of shear stress, representing between 19-42% lower shear stress than the unimproved bone reconstruction plate.

### Small Fragment Bone Reconstruction Plate

Two versions of a small fragment bone reconstruction plate measuring 72 mm×10.2 mm×3 mm and having six 4.4 mm outer diameter attachment bores (compatible with 4.0 mm diameter screws) were each tested using the two load cases as described above, the improved plate including ridges 122 forming a thread pattern within each of the attachment bores, the thread pattern operable to engage a 4.0 mm diameter screw. As shown in Table II of FIG. 2B, the top surface of the improved plate exhibited between 354 MPa and 358 MPa of tensile stress and the bottom surface of the improved plate exhibited between 303 MPa and 309 MPa of compressive stress, these ranges representing generally 2% lower stress factors than the unimproved bone reconstruction plate. Regarding the second load test, the improved plate exhibited between 7.4 MPa and 23.6 MPa of shear stress, representing between 7-20% lower shear stress than the unimproved bone reconstruction plate.

### Basic Fragment Bone Reconstruction Plate

Two versions of a basic fragment bone reconstruction plate measuring 94 mm×12 mm×4.1 mm and having six 5.6 mm outer diameter attachment bores (compatible with 4.0 mm diameter screws) were each tested using the two load cases as described above, the improved plate including ridges 122 forming a thread pattern within each of the attachment bores, the thread pattern operable to engage a 4.0 mm diameter screw. As shown in Table III of FIG. 2C, the top surface of the improved plate exhibited between 121 MPa and 124 MPa of tensile stress and the bottom surface of the improved plate exhibited between 112 MPa and 114 MPa of compressive stress, these ranges representing generally 34% and 42% lower stress factors, respectively, than the unimproved bone reconstruction plate. Regarding the second load test, the improved plate exhibited between 2.6 MPa and 12.4 MPa of shear stress, representing between 41-61% lower shear stress than the unimproved bone reconstruction plate.

In summary, it may be seen as one aspect of the present invention that a bone reconstruction plate 100 defining one or more attachment bores 120 includes one or more ridges 122 disposed within the one or more attachment bores 120. The ridges 122 are operable to provide increased resistance to fatigue failure around the attachment bores 120 of the bone reconstruction plate 100 where fatigue failure is most often observed.

It should be noted that the term “comprising” does not exclude other features, and the definite article “a” or “an” does not exclude a plurality, except when indicated. It is to be further noted that elements described in association with different embodiments may be combined.

The foregoing description has been presented for purposes of illustration and description. It is not intended to be exhaustive or to limit the invention to the precise form disclosed, and obviously many modifications and variations are possible in light of the disclosed teaching. The described embodiments were chosen in order to best explain the principles of the invention and its practical application to thereby enable others skilled in the art to best utilize the invention in various embodiments and with various modifications as are suited to the particular use contemplated. It is intended that the scope of the invention be defined solely by the claims appended hereto.

1. A bone reconstruction plate comprising a longitudinally extending plate defining one or more attachment bores, the longitudinally extending plate including ridges extending into one or more attachment bores, the one or more ridges operable to provide increased resistance to fatigue failure around the attachment bores of the bone reconstruction plate.

2. The bone reconstruction plate of claim 1, wherein the one or more ridges includes a thread pattern disposed within the attachment bore, the thread pattern adapted to engage a screw for securing the bone reconstruction plate onto a bone.

3. The bone reconstruction plate of claim 1, wherein said bone reconstruction plate further includes a top side and a bottom side of the longitudinally extending plate, wherein the bone plate measures 76 mm×8.8 mm×2 mm.

4. The bone reconstruction plate of claim 3, wherein under a combined compression and bending load, the bone reconstruction plate exhibits between 774 MPa and 795 MPa of tensile stress and between 534 MPa and 539 MPa of compressive stress.

5. The bone reconstruction plate of claim 3, wherein, under a torsion load, the top and bottom longitudinal sides of said bone reconstruction plate exhibit between 12 MPa and 45 MPa of shear stress.

6. The bone reconstruction plate of claim 1, wherein said bone reconstruction plate further includes a top side and a bottom side of the longitudinally extending plate, wherein the bone plate measures 72 mm×10.2 mm×3 mm.

7. The bone reconstruction plate of claim 6, wherein, under a combined compression and bending load, the top longitudinal side of said bone reconstruction plate exhibits between 354 MPa and 356 MPa of tensile stress, and the bottom longitudinal side of said bone reconstruction plate exhibits between 303 MPa and 309 MPa of compressive stress.

8. The bone reconstruction plate of claim 6, wherein, under a torsion load, the top and bottom longitudinal sides of said bone reconstruction plate exhibit between 7.5 MPa and 23.6 MPa of shear stress.

9. The bone reconstruction plate of claim 1, wherein said bone reconstruction plate further includes a top side and a bottom side of the longitudinally extending plate, wherein the bone plate measures 94 mm×12 mm×4.1 mm.

10. The bone reconstruction plate of claim 9, wherein, under a combined compression and bending load, the top longitudinal side of said bone reconstruction plate exhibits between 121 MPa and 124 MPa of tension stress, and the bottom longitudinal side of said bone reconstruction plate exhibits between 112 MPa and 114 MPa of compression stress.

11. The bone reconstruction plate of claim 9, wherein, under a torsion load, the top and bottom longitudinal side of said bone reconstruction plate exhibits between 2.6 MPa and 12.4 MPa of shear stress.
